(19) United States
(12) Patent Application Publication
(10) Pub. No.: US 2025/0268241 A1
(43) Pub. Date: Aug. 28, 2025

(54) SYSTEM FOR KEEPING LIVE INSECTS OR ANIMALS, AND METHOD

(71) Applicant: Spectrum Brands, Inc., Middleton, WI (US)

(72) Inventors: Joe Flora, Simi Valley, CA (US); Chad Robbins, Middleton, WI (US)

(21) Appl. No.: 19/061,675

(22) Filed: Feb. 24, 2025

Related U.S. Application Data
(60) Provisional application No. 63/558,202, filed on Feb. 27, 2024.

(51) Int. Cl. A01K 67/34

(52) U.S. Cl. CPC A01K 67/34

(57) ABSTRACT

A system for keeping live insects or animals. The system has a shelf assembly having at least a bottom wall and a rear wall. The bottom wall has opposite interior and exterior bottom wall surfaces and an outer perimeter with a rear face; the rear wall being mounted to extend upwardly from the rear face of the bottom wall. The rear wall having opposite interior and exterior rear wall surfaces. An airflow plenum is along the interior rear wall surface. The plenum has an airflow opening arrangement. A powered fan is in airflow communication with the plenum.

### CROSS-REFERENCE TO RELATED APPLICATIONS

This application claims priority to U.S. provisional patent application 63/558,202, filed on Feb. 27, 2024; the disclosure of which is incorporated herein by reference in its entirety.

### TECHNICAL FIELD

This disclosure relates to systems and arrangements for keeping live insects or animals, such as in a retail setting. In particular, this disclosure relates to an arrangement having ventilation to aid in sustaining life of insects or animals in retail settings, and methods of their use.

### BACKGROUND

Some people keep reptiles, such as snakes or lizards as pets. Birds, rats, and frogs are also kept as pets. Many reptiles, birds, frogs, and rats have live insects, such as crickets, as part of their diet. Retail locations specializing in pets and pet food can find it helpful to sell live insects, such as crickets, to customers who keep pets that would eat insects, such as crickets.

One problem is how to keep a large quantity of insects/crickets available for sale and in a convenient and easily accessible location in the retail setting, while also preventing fatality of them. Improvements are desirable.

### SUMMARY

In general, a system for keeping live insects or animals is provided that improves the prior art.

In one aspect, a system for keeping live insects or animals is provided. The system comprises: a shelf assembly having at least a bottom wall and a rear wall; the bottom wall having opposite interior and exterior bottom wall surfaces and an outer perimeter with a rear face; the rear wall being mounted to extend upwardly from the rear face of the bottom wall; the rear wall having opposite interior and exterior rear wall surfaces; an airflow plenum along the interior rear wall surface; the plenum having an airflow opening arrangement; and a powered fan in airflow communication with the plenum.

In example embodiments, there further includes a plurality of support members holding the bottom wall away from a ground surface.

In some embodiments, the plenum is mounted against the interior bottom wall surface; the bottom wall includes a fan-holding region; the fan-holding region being within the plenum; and the powered fan is held within the fan-holding region.

In some arrangement, the support members are legs.

In examples, the support members may be casters.

In example embodiments, the outer perimeter of the bottom wall has a front face opposite of the rear face, and first and second side faces extending between the front face and rear face; the shelf assembly further includes a first side wall and a second side wall; the first side wall being along the first side face of the bottom wall; and the second side wall being along the second side face of the bottom wall.

In some arrangements, there further includes a slidable panel mounted to slide over the bottom wall and away from the rear wall.

Some embodiments further include a portable first bin; the first bin including an access opening and a vent opening; the first bin being positionable in the shelf assembly with the vent opening in communication with the airflow opening arrangement of the plenum.

In examples, the shelf assembly further includes a first shelf member mounted against the interior rear wall surface, and spaced above the interior bottom wall surface a sufficient distance to accommodate a bin between the first shelf member and bottom wall; and the shelf assembly defines a lower bin holding region between the first shelf member and bottom wall; and an upper bin holding region above the first shelf member.

In some arrangements, the plenum extends along the interior rear wall surface to extend beyond the first shelf member into the upper bin holding region; and the airflow opening arrangement in the plenum includes at least a lower opening in the lower bin holding region, and an upper opening in the upper bin holding region.

Some embodiments further include a portable second bin; the second bin including an access opening and a vent opening; the second bin being positionable in the upper bin holding region with the vent opening of the second bin in communication with the upper opening of the plenum.

In some examples, the shelf system further includes a top wall extending at least partially over the upper bin holding region.

Example arrangements can further include a peg board back extending from the rear wall and above the top wall.

In some systems, the shelf assembly defines a double bin holding region along the bottom wall and between the first side wall and second side wall; the double bin holding region being sized to accommodate a portable first bin and a portable second bin.

In some embodiments, the airflow opening arrangement in the plenum includes at least a first opening and a second opening; the first opening and second opening being spaced laterally apart; the first opening positioned to be in communication with the portable first bin, and the second opening positioned to be in communication with the portable second bin.

Some example arrangement include the portable first bin including an access opening and a vent opening positioned to be in communication with the first airflow opening arrangement of the plenum; and the portable second bin including an access opening and a vent opening positioned to be in communication with the second airflow opening arrangement of the plenum.

The shelf system can further include a top wall extending at least partially over the double bin holding region.

The system can further include a peg board back extending from the rear wall and above the top wall.

Some embodiments further including a bag holder secured to the shelf system.

In some arrangements, the first bin includes a holding section defining an interior and a transparent lid moveably attached to the holding section; the first bin having a plurality of vent openings in the holding section.

In some embodiments, the powered fan moves between 30-50 cu. ft/minute.

In another aspect a method of keeping live insects or animals is provides. The method comprising: providing a shelf assembly having at least a bottom wall and a rear wall; the bottom wall having opposite interior and exterior bottom wall surfaces and an outer perimeter with a rear face; the rear wall being mounted to extend upwardly from the rear face of the bottom wall; the rear wall having opposite interior and exterior rear wall surfaces; providing an airflow plenum along the interior rear wall surface; the plenum having an airflow opening arrangement; and using a fan to provide air into the plenum.

The method can further include positioning a portable first bin having a vent opening in the shelf assembly with the vent opening in communication with the airflow opening arrangement of the plenum.

In some example methods, the shelf assembly further includes a first shelf member mounted against the interior rear wall surface, and spaced above the interior bottom wall surface a sufficient distance to accommodate a bin between the first shelf member and bottom wall; the shelf assembly defines a lower bin holding region between the first shelf member and bottom wall; and an upper bin holding region above the first shelf member; the plenum extends along the interior rear wall surface to extend beyond the first shelf member into the upper bin holding region; and the airflow opening arrangement in the plenum includes at least a lower opening in the lower bin holding region, and an upper opening in the upper bin holding region.

The method may further include providing a portable second bin; the second bin including a vent opening; and positioning the second bin in the upper bin holding region with the vent opening of the second bin in communication with the upper opening of the plenum.

In some examples, the shelf assembly defines a double bin holding region along the bottom wall and between the first side wall and second side wall; and the method can include positioning a portable first bin and a portable second bin in the double bin holding region.

In some arrangements, the airflow opening arrangement in the plenum includes at least a first opening and a second opening; the first opening and second opening being spaced laterally apart; the first opening positioned to be in communication with the portable first bin, and the second opening positioned to be in communication with the portable second bin.

The method may further include putting insects in the portable first bin and portable second bin.

A variety of examples of desirable features or methods are set forth in part in the description that follows, and in part will be apparent from the description, or may be learned by practicing various aspects of the disclosure. The aspects of the disclosure may relate to individual features as well as combinations of features. It is to be understood that both the foregoing general description and the following detailed description are explanatory only, and are not restrictive of the claimed invention.

### BRIEF DESCRIPTION OF THE DRAWINGS

FIG. 1 is a perspective view of a first embodiment of a shelf assembly system for keeping live insects or animals, constructed in accordance with principles of this disclosure;

FIG. 2 is a front view of the shelf assembly system of FIG. 1;

FIG. 3 is a side view of the shelf assembly system of FIG. 1;

FIG. 4 is a perspective view of a portable bin, used with the system of FIG. 1;

FIG. 5 is a partial, bottom, perspective view of the shelf assembly system of FIG. 1;

FIG. 6 is a perspective view of a second embodiment of a shelf assembly system for keeping live insects or animals, constructed in accordance with principles of this disclosure;

FIG. 7 is a front view of the shelf assembly system of FIG. 6;

FIG. 8 is a side view of the shelf assembly system of FIG. 6;

FIG. 9 is a partial, bottom, perspective view of the shelf assembly system of FIG. 6; and

FIG. 10 is a perspective view of the shelf assembly of FIG. 1, but with the bin removed from the lower bin holding region.

### DETAILED DESCRIPTION

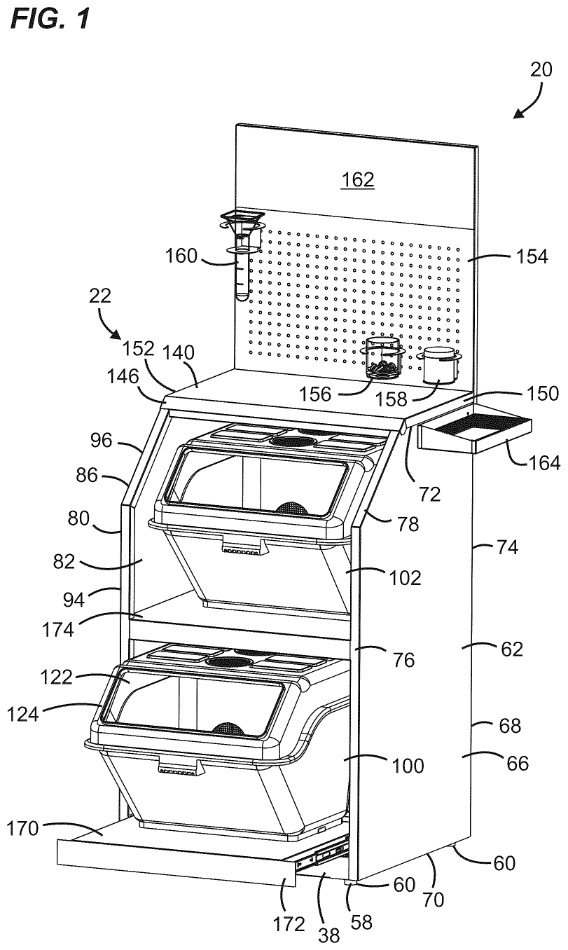

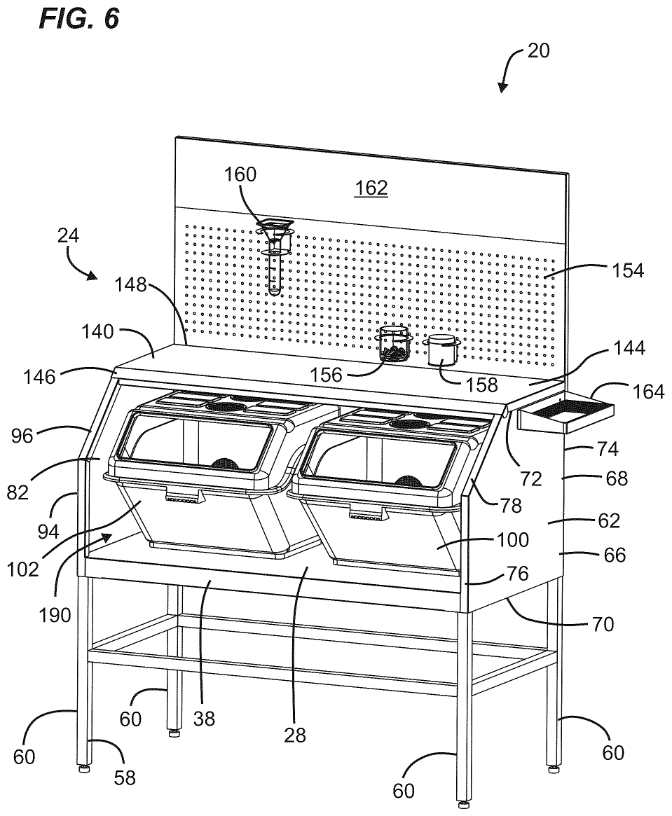

FIGS. 1 and 6 depict two example embodiments of a system for keeping live insects or animals; the system shown generally at 20. The system 20 of FIG. 1 is a stacked shelf system 22, while the system 20 of FIG. 6 is a side-by-side shelf system 24.

The system 20 includes a shelf assembly 26. By the term “shelf assembly”, it is meant a unit of components fitted, connected, fastened, or formed together having at least one or more rigid surfaces for the storage or display of one or more objects. The rigid surface is typically mostly flat and planar, but can have some variations.

The shelf assembly 26 includes a bottom wall 28. The bottom wall 28 is a rigid material, often sheet metal, or a hard plastic, or wood, which is located adjacent to or against a ground or floor. The bottom wall 28 has an interior facing wall surface 30, an opposite exterior facing wall surface 32, and an outer perimeter 34 surrounding the interior wall surface 30 and exterior wall surface 32.

In the example embodiment shown, the interior wall surface 30 and exterior wall surface 32 are generally flat, and planar. The outer perimeter 34 is rectangular in shape, but could be other shapes. In this example, the rectangular outer perimeter has a rear face 36, opposite front face 38, a first side face 40, and a second side face. The first side face 40 and second side face extend between the front face 38 and rear face 36.

The shelf assembly 26 further includes a rear wall 44. The rear wall 44 is a rigid material, often sheet metal, or a hard plastic, or wood, which is mounted to extend upwardly from the rear face 36 of the bottom wall 28. The rear wall 44 has an interior facing rear wall surface 46 and an opposite exterior facing rear wall surface 48. In general, the interior rear wall surface 46 and exterior rear wall surface 48 are flat and planar. The rear wall 44 is rectangular in shape, but could be other shapes.

The system 20 further includes an airflow channel or plenum 50. The plenum 50 is along the interior rear wall surface 46 and includes an airflow opening arrangement 52.

In one example embodiment, the plenum 50 is mounted against the interior bottom wall surface 30. The plenum 50 is constructed and arranged to allow for the flow of air therethrough.

The system 20 further includes a powered fan 54. The fan 54 is positioned to be in airflow communication with the plenum 50 to draw the flow of air through the plenum 50. In one example, the powered fan moves between 30-50 cu. ft/minute, with enough power to circulate air, but with not so much power that it disturbs the insects/animals held in the system 20.

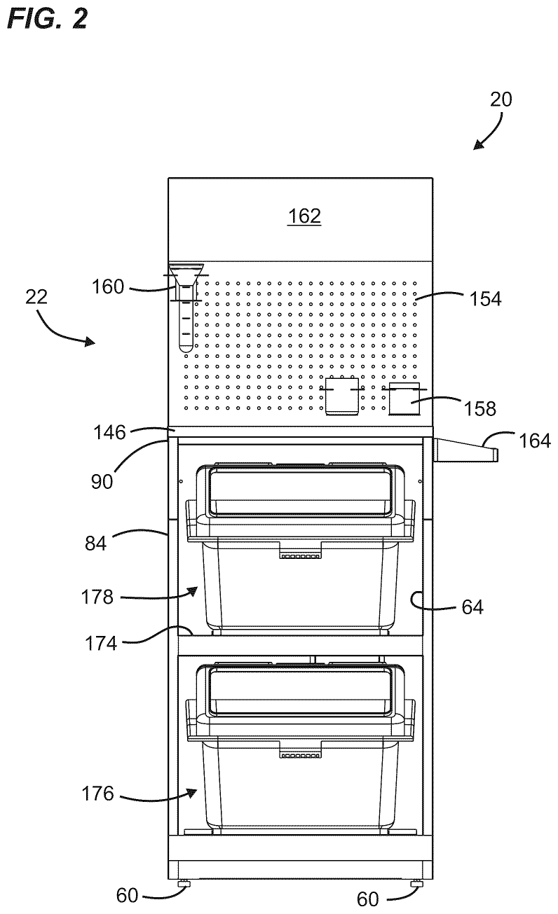

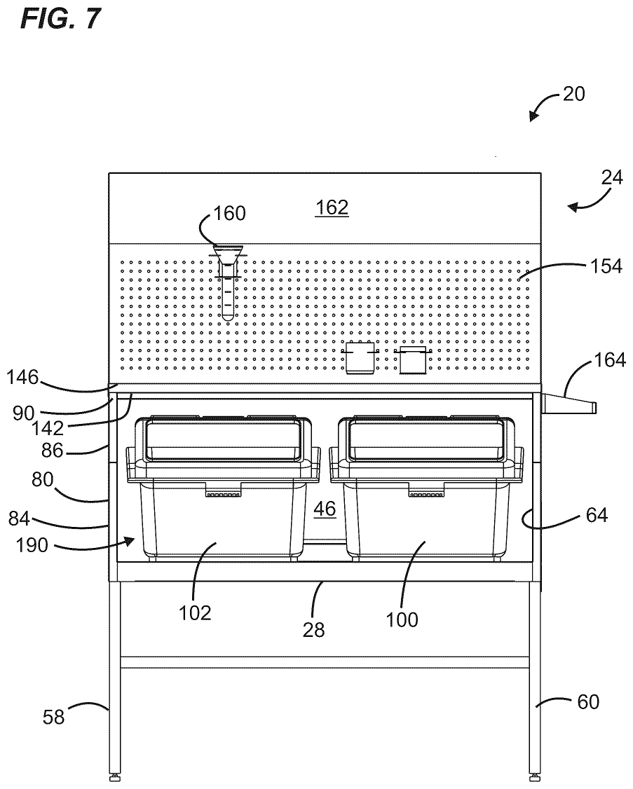

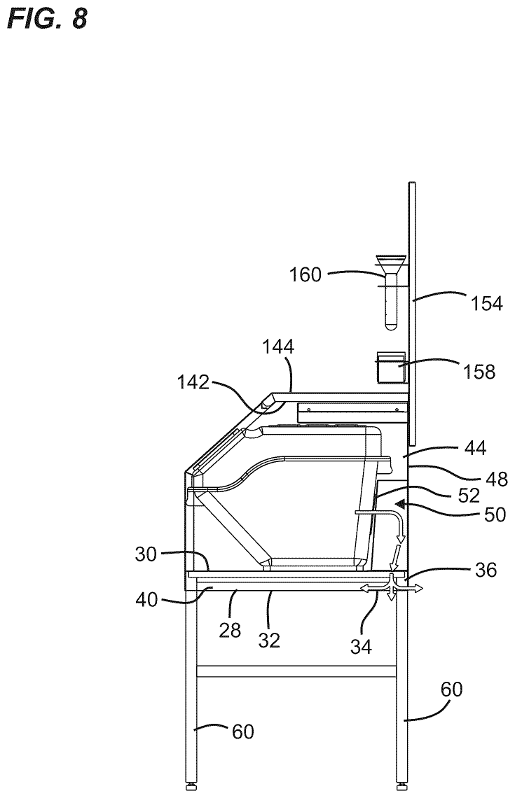

In example arrangements, the bottom wall 28 includes a fan-holding region 56. The fan-holding region 56 is located within the interior of the plenum 50 and is sized to hold the fan 54. The fan 54 is operably held within the fan-holding region 56. More details of the plenum 50 and fan 54 are discussed further below, in connection with the embodiment of FIGS. 1-5 and the embodiment of FIGS. 6-9.

The system 20 further includes a plurality of support members 58. The support members 58 hold the bottom wall 28 away from a ground surface. The support members 58 can be legs 60, including short legs 60 (FIG. 1) or longer legs 60 (FIG. 6). In other arrangements, the support members 58 can be casters.

The shelf assembly 26 further includes a first side wall 62. The first side wall 62 has an interior-facing surface 64, and an opposite exterior-facing surface 66. The first side wall 62 has a perimeter edge 68. The perimeter edge 68 can form many different shapes, and in the examples shown, the perimeter edge 68 is trapezoidal in shape. The perimeter edge 68 has a bottom edge 70, which opposes or faces the ground surface; an opposite top edge 72; a rear edge 74; an opposite front edge 76; and an angled edge 78 extending between the front edge 76 and top edge 72. The first side wall 62 a rigid material, often sheet metal, or a hard plastic, or wood.

The shelf assembly 26 further includes a second side wall 80. The second side wall 80 has an interior-facing surface 82, and an opposite exterior-facing surface 84. The second side wall 80 has a perimeter edge 86. The perimeter edge 86 can form many different shapes, and in the examples shown, the perimeter edge 86 is trapezoidal in shape. The perimeter edge 86 has a bottom edge 88, which opposes or faces the ground surface; an opposite top edge 90; a rear edge; an opposite front edge 94; and an angled edge 96 extending between the front edge 94 and top edge 90. The second side wall 80 a rigid material, often sheet metal, or a hard plastic, or wood.

The first side wall 62 and the second side wall 80 are positioned to be against opposite faces of the bottom wall 28, such that the interior facing surfaces 64, 82 are opposing each other. That is, the first side wall 62 is along the first side face 40 of the bottom wall 28, while the second side wall 80 is along the second side face 42 of the bottom wall 28.

The shelf assembly 26 further includes a top wall 140. The top wall 140 has an inner facing surface 142 and an opposite exterior facing surface 144. In the example embodiment, the top wall 140 has a rectangular perimeter edge including a front edge 146, rear edge 148, and first and second side edges 150, 152. The top wall 140 extends horizontally between the first side wall 62 and second side wall 80. The inner facing surface 142 faces toward the remainder of the shelf assembly 26. The first and second side edges 150, 152 are adjacent to the top edges 72, 90 of the first side wall 62 and second side wall 80.

The system 20 can further include an optional pegboard back 154. The pegboard back 154 extends from the rear wall 44 and above the top wall 140. The pegboard back 154 is useable for holding useful items, such as rubber band containers 156, calcium powder containers 158, a holder for a cricket counter 160, etc. A section 162 can also be used to hold signage.

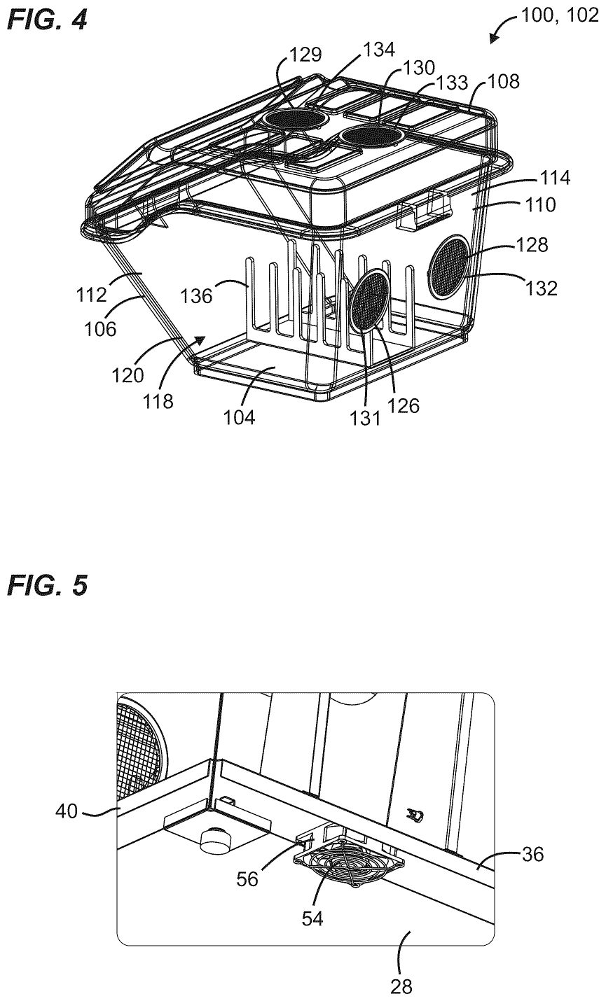

The system 20 further includes a portable bin for holding live insects or animals. In the example embodiments shown, there is a portable first bin 100 and a portable second bin 102. The system 20 can be designed to accommodate more bins, as well. Both bins 100, 102 have the same features, so in describing the example embodiment shown in FIG. 4, it should be understood that both bins 100, 102 are being described.

The bins 100, 102 have a base 104, a surrounding wall 106 extending up from the base 104, and a cover 108 secured on top of the surrounding wall 106. The surrounding wall 106 includes a rear wall 110, first and second side walls 112, 114, and a front wall 116. The front wall 116 angles outwardly from the base 104 at a non-perpendicular angle to project away from the base 104. The base 104, surrounding wall 106, and cover 108 enclose an interior 118, which functions as a holding section 120 for holding crickets, insects, or other live animals.

The cover 108 includes a transparent lid 122. The lid 122 allows for viewing into the holding section 120. The lid 122 is moveably attached to the holding section 120, and in the example shown, is pivotably attached to a remainder of the cover 108. The lid 122 pivotably covers an access opening 124, providing access to the interior 118. A customer can view the interior 118 of the bin 100, 102 through the lid 122, and then pivot the lid 122 to expose the access opening 124. When the access opening 124 is exposed, the customer can reach into the interior 118 and remove crickets, insects, or other live animals being kept in the holding section 120 of the bins 100, 102.

The bins 100, 102 each has at least one vent opening 126. The at least one vent opening 126 is located such that, when the bins 100, 102 are positioned on the shelf assembly 26, the vent opening 126 is put into airflow communication with the airflow opening arrangement 52 of the plenum 50.

In the example embodiment illustrated, the vent opening 126 is in the rear wall 110 of the bin 100, 102. This way, the bin 100, 102 can be positioned on the shelf assembly 26 with its rear wall 110 against the shelf assembly rear wall 44, and with the transparent lid 122 facing outward from the shelf assembly 26.

In preferred embodiments, each of the bins 100, 102 has a plurality of vent openings 128, 129, 130. The vent openings 128, 129, 130 are in the holding section 120. In the example shown, the rear wall 110 has vent opening 126 and vent opening 128, spaced laterally apart from each other, and centered between the top and bottom of the bin 100, 102. The cover 108 has vent openings 129, 130 in a portion of the cover 108 that is spaced over and generally parallel to the base 104. There can be many variations, and include more or fewer vent openings.

The vent openings 126, 128, 129, 130 are depicted as round, but could be other shapes. The openings 126, 128, 129, 130 are covered with a screen 131, 132, 133, 134.

The bins 100, 102 can be made from plastic or other materials, such as metal or wood. FIG. 4 allows for viewing the interior 118, but it should be understood that the surrounding wall 106 is typically opaque and not transparent. In the interior 118, optional egg carton holders 136 are visible, resting against the base 104. The egg carton holders 136 are used to hold egg cartons (not shown), which make for hospitable homes for crickets (or other insects) and provide for extra space in the interior 118.

An optional bag holder 164 is secured to the shelf assembly 26. In the example shown, the bag holder 164 is attached to the exterior facing surface 66 of the first side wall 62 to project therefrom. The bag holder 164 holds bags for packaging the crickets, insects, or other animals held in the bins 100, 102.

### Stacked Shelf System, FIGS. 1-3

In reference now to the stacked shelf system 22 of FIGS. 1-3, the system 22 further includes a slidable panel 170. The panel 170 is movably mounted to slide over the bottom wall 28 and toward/away from the rear wall 44. The panel 170 extends horizontally between the first side wall 62 and second side wall 80. The panel 170 includes a hand hold 172 at a free end along a front edge, which allows for convenient grasping by a human hand. The panel 170 is sized to hold one of the bins 100, 102. In use, a person can grasp the hand hold 172 to pull the panel 170 out from a remaining part of the shelf system 22 to allow for better access to the bin 100, 102 resting on the panel 170.

The shelf assembly 26 further includes a first shelf member 174. In the example illustrated, the first shelf member 174 is mounted against the interior rear wall surface 46 and spaced above the interior bottom wall surface 30 a sufficient distance to accommodate a bin between the first shelf member 174 and the bottom wall 28. The first shelf member 174 extends horizontally between the first side wall 62 and the second side wall 80.

The shelf assembly 26 defines a lower bin holding region 176 between the first shelf member 174 and bottom wall 28. In particular, the lower bin holding region 176 is between the first shelf member 174 and the slidable panel 170. The lower bin holding region 176 is sized to accommodate one of the bins 100, 102.

The shelf assembly 26 defines an upper bin holding region 178 above the first shelf member 174. The top wall 140 extends at least partially over the upper bin holding region 178. The upper bin holding region 178 is sized to accommodate one of the bins 100, 102.

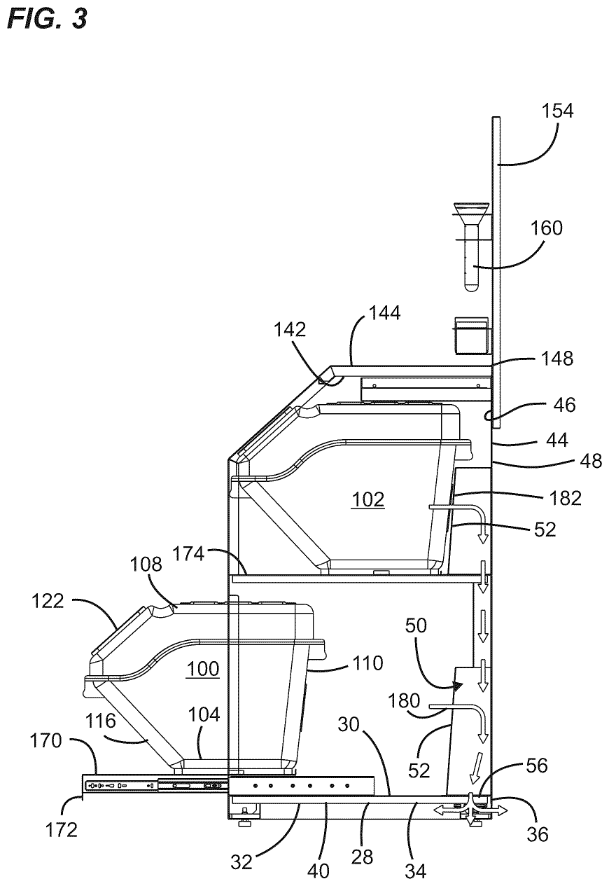

In reference now to FIG. 3, the plenum 50 extends along the interior rear wall surface 46 to extend beyond the first shelf member 174 into the upper bin holding region 178. The airflow opening arrangement 52 in the plenum 50 includes at least a lower opening 180 in the lower bin holding region 176, and an upper opening 182 in the upper bin holding region 178.

The portable first bin 100 is positionable in the lower bin holding region 176, such that the vent opening 126 can be against and in direct airflow communication with the lower opening 180 of the plenum 50. The portable second bin 102 is positionable in the upper bin holding region 178, such that the vent opening 126 of the second bin 102 can be against and in direct airflow communication with the upper opening 182 of plenum 50.

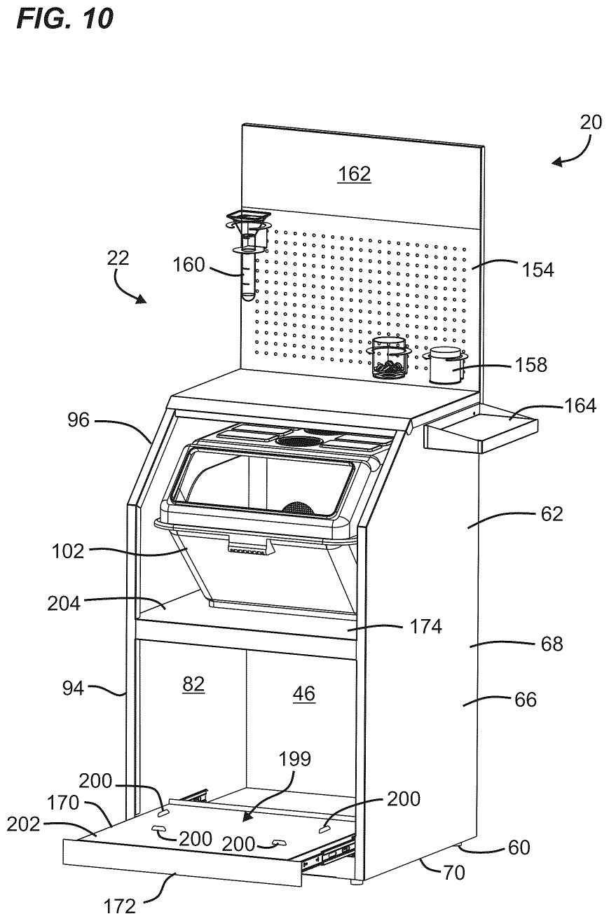

The system 20 includes an aid 199 to the align the vents 126 in the bins 100, 102 with the openings 180, 182 in the plenum 50. In FIG. 10, the aid is embodied as a plurality of projecting tabs 200, arranged to project from an upper face 202 of the slidable panel 170 and from an upper face 204 of the first shelf member 174. The tabs 200 are located such that an outside perimeter of the base 104 of the bins 100, 102 is closely contained within the tabs 200. The tabs 200 can be small, sheet metal tabs projections.

In reference now to FIG. 3, when the fan 54 is operating, air is drawn through the bins 100, 102, and from the bins through vent opening 126, then through the respective lower opening 180 or upper opening 182, through the plenum 50 and out through the airflow opening arrangement 52.

### Side by Side Shelf System, FIGS. 6-9

In reference now to FIGS. 6-9, the side by side shelf system 24 defines a double bin holding region 190 along the bottom wall 28 and between the first side wall 62 and second side wall 80. The top wall 140 extends at least partially over the double bin holding region 190. The double bin holding region 190 is sized to accommodate the portable first bin 100 and portable second bin 102.

Attention is directed to FIG. 9. The airflow opening arrangement 52 in the plenum 50 includes at least a first opening 192 and a second opening 194. The first opening 192 and second opening 194 are spaced laterally apart. The first opening 192 is positioned to be in communication with the portable first bin 100, and the second opening 194 is positioned to be in communication with the portable second bin 102.

The side by side shelf system 24 includes an aid to the align the vents 126 in the bins 100, 102 with the openings 192, 194 in the plenum 50. The aid 199 is embodied as projecting tabs 200, arranged to project from an upper face of the bottom wall 28, analogous to the tabs 200 shown in FIG. 10, projecting from the slidable panel 170 and from the first shelf member 174. In the side by side shelf system 24, there are two sets of tabs 200. A first of the two sets of tabs 200 projects from the bottom wall 28 to outline where the portable first bin 100 is positioned, and a second of the two sets of tabs 200 projects from the bottom wall 28 to outline where the portable second bin 102 is positioned.

For example, the portable first bin 100 is positioned in the double bin holding region 190 such that the vent opening 126 is against the first opening 192 of the plenum 50 to be in airflow communication therewith. The portable second bin 102 is positioned in the double bin holding region 190 such that the vent opening 126 of the second bin 102 is against the second opening 194 of the plenum 50 to be in airflow communication therewith.

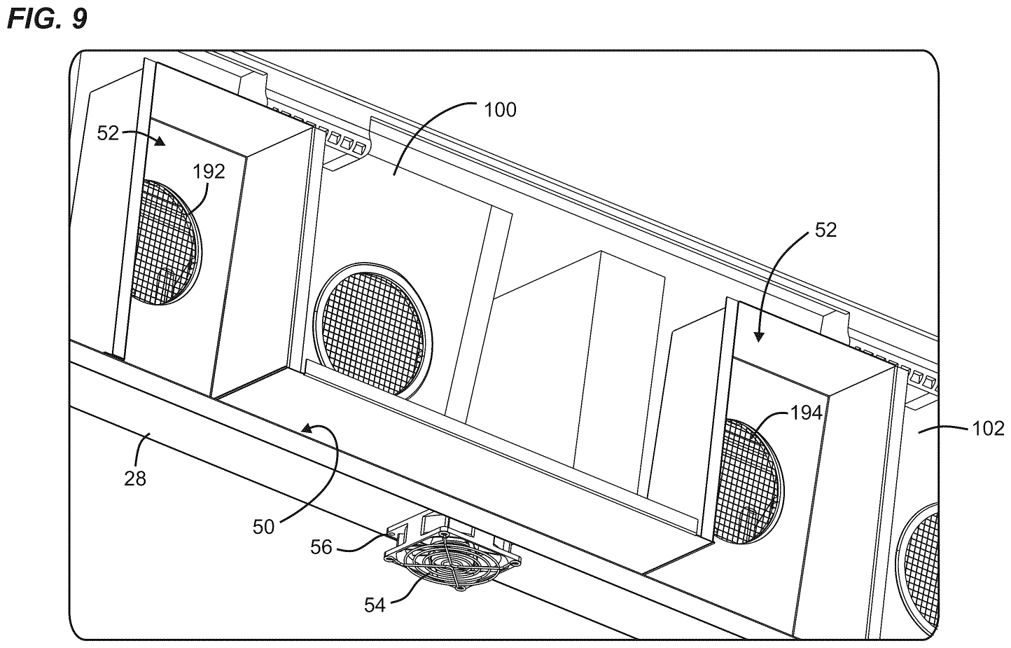

In reference to FIG. 8, when the fan 54 is operating, air is drawn through the bins 100, 102, and from the bins through vent opening 126, then through the respective first opening 192 or second opening 194, through the plenum 50 and out through the airflow opening arrangement 52.

### Example Methods of Use

The above system 20 is usable in a method of keeping live insects or animals. The method includes providing the shelf assembly 26 having at least bottom wall 28 and rear wall 44. The bottom wall 28 has opposite interior and exterior bottom wall surfaces 30, 32 and outer perimeter 34 with rear face 36. The rear wall 44 is mounted to extend upwardly from the rear face 36 of the bottom wall 28. The rear wall 44 has opposite interior and exterior rear wall surfaces 46, 48.

The method includes providing airflow plenum 50 along the interior rear wall surface 46. The plenum 50 has airflow opening arrangement 52.

The method includes using fan 54 to provide air into the plenum 50.

The method further includes positioning portable first bin 100 having vent opening 126 in the shelf assembly 26 with the vent opening 126 in communication with the airflow opening arrangement 52 of the plenum 50.

The method further includes providing portable second bin 102 having vent opening 126 and positioning the second bin 102 in the upper bin holding region 178 with the vent opening 126 of the second bin 102 in communication with the upper opening of the plenum 182.

In one example method, the shelf assembly 26 defines double bin holding region 190 along the bottom wall 28 and between the first side wall 62 and second side wall 80. The method includes positioning portable first bin 100 and portable second bin 102 in the double bin holding region 190. The airflow opening arrangement 52 in the plenum 50 includes at least first opening 192 and second opening 194. The first opening 192 and second opening 194 are spaced laterally apart. The first opening 192 is positioned to be in communication with the portable first bin 100, and the second opening 194 is positioned to be in communication with the portable second bin 102.

The method can further including putting insects in the portable first bin 100 and portable second bin 102. The method can further include removing insects from the first bin 100 or second bin 102. These steps can include pivoting the lid 122 to expose the access opening 124 into the interior 118 of the bin 100, 102.

The method can further include grasping hand hold 172, sliding the panel 170 out and away from the rear wall 44 and then accessing the bin 100, 102 in the lower bin holding region 176.

The above represents example principles. Many embodiments can be made using these principles.

What is claimed is:

1. A system for keeping live insects or animals; the system comprising:
(a) a shelf assembly having at least a bottom wall and a rear wall;
(i) the bottom wall having opposite interior and exterior bottom wall surfaces and an outer perimeter with a rear face;
(ii) the rear wall being mounted to extend upwardly from the rear face of the bottom wall; the rear wall having opposite interior and exterior rear wall surfaces;
(b) an airflow plenum along the interior rear wall surface; the plenum having an airflow opening arrangement; and
(c) a powered fan in airflow communication with the plenum.

2. The system of claim 1 further including a plurality of support members holding the bottom wall away from a ground surface.

3. The system of claim 2, wherein:
(a) the plenum is mounted against the interior bottom wall surface;
(b) the bottom wall includes a fan-holding region;
(i) the fan-holding region being within the plenum; and
(c) the powered fan is held within the fan-holding region.

4. The system of claim 2, wherein the support members are legs.

5. The system of claim 2, wherein the support members are casters.

6. The system of claim 1, wherein:
(a) the outer perimeter of the bottom wall having a front face opposite of the rear face, and first and second side faces extending between the front face and rear face;
(b) the shelf assembly further includes a first side wall and a second side wall;
(i) the first side wall being along the first side face of the bottom wall; and
(ii) the second side wall being along the second side face of the bottom wall.

7. The system of claim 6 further including a slidable panel mounted to slide over the bottom wall and away from the rear wall.

8. The system of claim 1, further including a portable first bin; the first bin including an access opening and a vent opening; the first bin being positionable in the shelf assembly with the vent opening in communication with the airflow opening arrangement of the plenum.

9. The system of claim 1, wherein:
(a) the shelf assembly further includes a first shelf member mounted against the interior rear wall surface, and spaced above the interior bottom wall surface a sufficient distance to accommodate a bin between the first shelf member and bottom wall; and
(b) the shelf assembly defines a lower bin holding region between the first shelf member and bottom wall; and an upper bin holding region above the first shelf member.

10. The system of claim 9 wherein:
(a) the plenum extends along the interior rear wall surface to extend beyond the first shelf member into the upper bin holding region; and
(b) the airflow opening arrangement in the plenum includes at least a lower opening in the lower bin holding region, and an upper opening in the upper bin holding region.

11. The system of claim 8, further including a portable second bin; the second bin including an access opening and a vent opening; the second bin being positionable in the upper bin holding region with the vent opening of the second bin in communication with the upper opening of the plenum.

12. The system of claim 9, wherein the shelf system further includes a top wall extending at least partially over the upper bin holding region.

13. The system of claim 12, further including a peg board back extending from the rear wall and above the top wall.

14. The system of claim 6, wherein the shelf assembly defines a double bin holding region along the bottom wall and between the first side wall and second side wall; the double bin holding region being sized to accommodate a portable first bin and a portable second bin.

15. The system of claim 14 wherein the airflow opening arrangement in the plenum includes at least a first opening and a second opening; the first opening and second opening being spaced laterally apart; the first opening positioned to be in communication with the portable first bin, and the second opening positioned to be in communication with the portable second bin.

16. The system of claim 15 further including:
(a) the portable first bin including an access opening and a vent opening positioned to be in communication with the first airflow opening arrangement of the plenum; and
(b) the portable second bin including an access opening and a vent opening positioned to be in communication with the second airflow opening arrangement of the plenum.

17. The system of claim 14, wherein the shelf system further includes a top wall extending at least partially over the double bin holding region.

18. The system of claim 17, further including a peg board back extending from the rear wall and above the top wall.

19. The system of claim 1, further including a bag holder secured to the shelf system.

20. The system of claim 8, wherein the first bin includes a holding section defining an interior and a transparent lid moveably attached to the holding section; the first bin having a plurality of vent openings in the holding section.

21. The system of claim 8, wherein the shelf assembly includes projecting tabs to aid in positioning the first bin with the vent opening in communication with the airflow opening arrangement of the plenum.

22. The system of claim 1, wherein the powered fan moves between 30-50 cu. ft/minute.

23. A method of keeping live insects or animals; the method comprising:
(a) providing a shelf assembly having at least a bottom wall and a rear wall;
(i) the bottom wall having opposite interior and exterior bottom wall surfaces and an outer perimeter with a rear face;
(ii) the rear wall being mounted to extend upwardly from the rear face of the bottom wall; the rear wall having opposite interior and exterior rear wall surfaces;
(b) providing an airflow plenum along the interior rear wall surface; the plenum having an airflow opening arrangement; and
(c) using a fan to provide air into the plenum.

24. The method of claim 23 further including:
positioning a portable first bin having a vent opening in the shelf assembly with the vent opening in communication with the airflow opening arrangement of the plenum.

25. The method of claim 24, wherein a step of positioning the portable first bin includes positioning the portable first bin within a set of projecting tabs in the shelf assembly.

26. The method of claim 24, wherein:
(a) the shelf assembly further includes a first shelf member mounted against the interior rear wall surface, and spaced above the interior bottom wall surface a sufficient distance to accommodate a bin between the first shelf member and bottom wall;
(b) the shelf assembly defines a lower bin holding region between the first shelf member and bottom wall; and an upper bin holding region above the first shelf member;
(c) the plenum extends along the interior rear wall surface to extend beyond the first shelf member into the upper bin holding region; and
(d) the airflow opening arrangement in the plenum includes at least a lower opening in the lower bin holding region, and an upper opening in the upper bin holding region.

27. The method of claim 26 further including providing a portable second bin; the second bin including a vent opening; and positioning the second bin in the upper bin holding region with the vent opening of the second bin in communication with the upper opening of the plenum.

28. The method of claim 24, wherein the shelf assembly defines a double bin holding region along the bottom wall and between the first side wall and second side wall; and positioning a portable first bin and a portable second bin in the double bin holding region.

29. The method of claim 28, wherein the airflow opening arrangement in the plenum includes at least a first opening and a second opening; the first opening and second opening being spaced laterally apart; the first opening positioned to be in communication with the portable first bin, and the second opening positioned to be in communication with the portable second bin.

30. The method of claim 27, further including putting insects in the portable first bin and portable second bin.
